(19) United States
(12) Patent Application Publication
(10) Pub. No.: US 2026/0114720 A1
(43) Pub. Date: Apr. 30, 2026

(54) ARTICULATING ENDOSCOPE AND METHODS OF USING THE SAME

(71) Applicant: The Regents of the University of Colorado, a body corporate, Denver, CO (US)

(72) Inventors: Mark E. Rentschler, Boulder, CO (US); Brennan Moeller, Rayne, LA (US); Brian Herrmann, Niwot, CO (US)

(73) Assignee: The Regents of the University of Colorado, a body corporate, Denver, CO (US)

(21) Appl. No.: 18/927,396

(22) Filed: Oct. 25, 2024

Related U.S. Application Data
(60) Provisional application No. 63/592,001, filed on Oct. 20, 2023.

(51) Int. Cl. A61B 1/227; A61B 1/00; A61B 1/008; A61B 1/05

(52) U.S. Cl. CPC A61B 1/227; A61B 1/00042; A61B 1/00066; A61B 1/00177; A61B 1/00179; A61B 1/00183; A61B 1/008; A61B 1/05

(57) ABSTRACT

Embodiments disclosed herein relate to an endoscopic device and a method to provide dynamic visualization of the middle ear. The endoscopic device includes a handle and a shaft extending therefrom. The shaft includes a rigid portion distal the handle and an articulating portion distal the rigid portion. The articulating portion is configured to articulate with respect to the rigid portion. The articulating portion includes an imaging sensor coupled thereto. The handle can include one or more actuators configured to cause the articulating portion to articulate.

### CROSS REFERENCE TO RELATED APPLICATION

The present application claims priority to U.S. Provisional Patent Application No. 63/592,001, which was filed on Oct. 20, 2023. The contents of the above-mentioned patent application are hereby incorporated by reference in its entirety.

### FIELD OF THE INVENTION

The present disclosure is directed to endoscopes for use in medical procedures. In particular, the present disclosure relates to endoscopes for use in visualizing small and hard to reach areas, like, for example, the middle ear.

### BACKGROUND OF THE INVENTION

Ear surgery is required to address chronic infection and cholesteatoma, with the latter being a buildup of epithelium within the middle ear cavity. Failure to treat cholesteatoma can lead to facial muscle weakness, chronic drainage (otorrhea), dizziness, and hearing loss. Traditional removal of chronic suppurative otitis and/or cholesteatoma includes making ear canal incisions to lateralize the tympanic membrane (ear drum) and visually accessing the middle ear cavity. Conventionally, floor-mounted microscopes or hand-held rigid endoscopes have been used to provide visualization of the middle ear. The middle ear is a small, delicate cavity, and visualization can be impaired due to age, unfavorable ear canal anatomy, the depth of disease within the middle ear, and limitations of conventional floor-mounted microscopes and rigid endoscopes.

In some cases, inadequate visualization of the middle ear necessitates a more invasive mastoidectomy. Conventionally, a mastoidectomy involves making a large incision behind the auricle (ear) of the patient and drilling away a portion of the pneumatized mastoid bone. The mastoidectomy may necessitate secondary surgeries and/or cause future hearing complications for the patient. For example, the mastoidectomy may have a negative postoperative impact on hearing, as well as create deleterious effects on gas exchange within the ear and a reduction in internal tympanic membrane support.

Accordingly, there is a need in the art for an endoscope that provides enhanced visualization during middle ear surgeries and, accordingly, reduces the instances of mastoidectomies. It is with these thoughts in mind, among others, that the articulating endoscope of the present disclosure was conceived.

### SUMMARY

Aspects of the present disclosure include an endoscopic device. The endoscopic device includes a handle and a shaft extending from the handle to a distal end. The shaft includes a rigid portion extending distally from the handle and an articulating portion extending distally from the rigid portion towards the distal end. The rigid portion defines a first longitudinal axis, and the articulating portion defines a second longitudinal axis. An imaging sensor is coupled to the articulating portion, at or near the distal end of the shaft. The articulating portion is configured to articulate about an axis of articulation that is substantially perpendicular to each of the first longitudinal axis and the second longitudinal axis.

In certain instances, the second longitudinal axis is co-linear with the first longitudinal axis when the articulating portion is not articulated. In certain instances, the second longitudinal axis is coplanar with the first longitudinal axis when the articulating portion is articulated.

In certain instances, the second longitudinal axis defines an angle of articulation with respect to the first longitudinal axis. In certain instances, the angle of articulation is up to approximately 90-degrees.

In certain instances, the shaft includes a living hinge between the rigid portion and the articulating portion such that the living hinge defines the axis of articulation.

In certain instances, the articulating portion is configured to rotate about the first longitudinal axis.

In certain instances, rotation of the articulating portion about the first longitudinal axis defines an angle of rotation, wherein the angle of rotation is up to 360-degrees.

In certain instances, the handle includes a first actuator and a second actuator. Actuating the first actuator can cause the articulating portion to articulate about the axis of articulation, and actuating the second actuator can cause the shaft to rotate about the first longitudinal axis.

In certain instances, the handle is ergonomically configured such that when the handle is gripped, the shaft extends downward, a first actuator is positioned for thumb-manipulation and a second actuator is positioned for forefinger-manipulation.

Aspects of the present disclosure include an endoscopic device adapted to be ergonomically gripped by a hand of a user. The hand can include a palm, a thumb, a forefinger, a middle finger, a ring finger and a little finger, with the four fingers and the thumb extending from the palm. The endoscopic device includes an ergonomic handle, a shaft extending distally from the ergonomic handle, and an imaging sensor coupled to the shaft. The ergonomic handle is configured to be received by the hand of the user, and the ergonomic handle including first and second actuators. The shaft extends distally from the ergonomic handle, and the shaft including a rigid portion and an articulating portion. The imaging sensor is coupled to the articulating portion of the shaft. The first actuator is configured to cause the articulating portion to articulate with respect to the rigid portion, and the second actuator is configured to cause the articulating portion to rotate about a longitudinal axis of the rigid portion. When the ergonomic handle is gripped by the hand of the user, the palm and at least two of the four fingers wrap around the ergonomic handle, the thumb is positioned to manipulate the first actuator, the forefinger is positioned to manipulate the second actuator, and the shaft extends downward away from the ergonomic handle and the first actuator.

In certain instances, the ergonomic handle includes a barrel portion and a tapered portion extending distally from the barrel portion. In certain instances, the barrel portion is configured to receive at least the forefinger, middle finger and thumb of the user thereon.

In certain instances, the barrel portion includes a first portion defining a first cross-sectional area and a second portion defining a second cross-sectional area that is smaller than the first cross-sectional area. In certain instances, the first portion is configured to receive the forefinger of the user thereon and the second portion is configured to receive the middle finger of the user thereon.

In certain instances, the articulating portion is configured to articulate about an axis of articulation that is substantially perpendicular to a longitudinal axis of the rigid portion.

In certain instances, the first actuator is on a first side of the ergonomic handle and the second actuator is on a second side of the ergonomic handle opposite the first side.

In certain instances, the first actuator is positioned for thumb-manipulation and the second actuator is positioned for forefinger-manipulation.

In certain instances, the first actuator includes a lever configured to receive the thumb of the user thereon such that the lever can be slidably actuated. In certain instances, the second actuator includes a wheel configured to receive the forefinger of the user thereon such that the wheel can be rotatably actuated.

In certain instances, the ergonomic handle defines a length and a barrel portion of the ergonomic handle defines a maximum diameter. In certain instances, the length is between approximately 15 cm and approximately 20 cm. In certain instances, the maximum diameter is between approximately 5.4 cm and approximately 6.2 cm.

Aspects of the present disclosure include a method using an endoscopic device to provide visualization during a middle ear surgery of patient. The method includes gripping a handle of the endoscopic device with a hand. The endoscopic device includes the handle, a shaft distally extending from the handle to a distal end of the shaft, and an imaging sensor at least near, if not on, the distal end. The shaft includes a rigid portion and an articulating portion distal the rigid portion. The handle includes first and second actuators. The first actuator is configured to cause the articulating portion to articulate with respect to the rigid portion, and the second actuator is configured to cause the articulating portion to rotate with respect to a longitudinal axis of the rigid portion. The method further includes inserting the distal end of the shaft into an ear canal of an ear of the patient. The method further includes actuating at least one of the first and second actuators on the handle to cause the articulating portion of the shaft to at least one of articulate or rotate.

In certain instances, gripping the handle of the endoscopic device with the hand results in a palm of the hand, a forefinger of the hand, and a middle finger of the hand all wrapping around the handle. In certain instances, gripping the handle of the endoscopic device with the hand results in a thumb of the hand positioned to manipulate the first actuator, the forefinger positioned to manipulate the second actuator, and the shaft extending downward away from the handle and the first actuator.

In certain instances, the method further includes advancing the distal end to a middle ear of the patient.

In certain instances, the method further includes viewing, via the imaging sensor, at least a portion of a middle ear of the patient during otolaryngology procedure.

### BRIEF DESCRIPTION OF THE DRAWINGS

FIG. 1 illustrates an endoscopic device providing visualization of the middle ear in a perspective view, in accordance with embodiments of the disclosure.

FIGS. 2A-2B illustrate a conventional floor-mounted microscope providing visualization of the middle ear in a perspective view and a more detailed perspective view, respectively.

FIGS. 3A-3B illustrate a conventional rigid endoscope providing visualization of the middle ear in a perspective view and a more detailed perspective view, respectively.

FIGS. 4A-4B illustrate the endoscopic device of FIG. 1 in a left-side view and a right-side view, respectively, in accordance with embodiments of the disclosure.

FIG. 5 illustrates the handle of the endoscopic device with a hand of a user in a left-side view.

FIG. 6 illustrate an internal cavity of the handle, with various components therein, in a left-side, perspective view.

FIG. 7 illustrate the internal cavity of the handle in a proximal-perspective view.

FIGS. 8A-8B illustrate the articulation actuator in an unactuated position (FIG. 8A) and an actuated position (FIG. 8B) in left-side views.

FIG. 8C illustrates the articulating portion of the shaft in an articulated position overlaying the articulating portion of the shaft in an unarticulated position.

FIG. 9 illustrates the distal end of a shaft, in accordance with embodiments of the disclosure.

FIG. 10 illustrates the distal end of a shaft, in accordance with embodiments of the disclosure.

FIG. 11 illustrates the distal end of a shaft, in accordance with embodiments of the disclosure.

FIG. 12 is a flowchart showing a method of using the endoscopic device.

FIG. 13 illustrates the field of view of the endoscopic device compared to a conventional rigid endoscope.

FIG. 14 illustrates the Spatial Frequency Response (SFR) comparison of 20 averaged samples from a 0° Karl Storz endoscope, an Ambu bronchoscope, and the endoscopic device.

FIG. 15 illustrates a visual representation of variables used in quantifying the advantage of the controllable degrees of freedom of the endoscopic device.

### DETAILED DESCRIPTION

Aspects of the present disclosure relate to an endoscope for dynamic middle ear surgical visualization. The endoscope includes an ergonomic handle and a shaft extending from the ergonomic handle. The shaft has a rigid section and an articulating section that can articulate with respect to the rigid section. The articulating section has two controllable degrees of freedom (DOF). That is, the articulating section can articulate with respect to the rigid section and/or rotate about the central axis of the rigid section. A camera, which provides visualization, is attached to the articulating section of the shaft. Due to the two controllable DOFs, the camera can be dynamically articulated off-axis and/or axially rotated by articulating and/or rotating the articulating section of the shaft.

The ergonomic handle includes controls for each of the two controllable DOFs.

An articulation control, which can be a lever configured to be slidably actuated by a thumb of the user, can be actuated such that the articulating section articulates with respect to the rigid section. A rotation control, which can be a wheel configured to be rotatably actuated by a finger of the user, can be actuated such that the articulating section rotates about the central axis of the rigid section.

The endoscope disclosed herein may provide several advantages over conventional systems. As one example, the endoscopic device provides the ability for greater manipulation of the field of view over conventional systems, which can reduce the instances that require mastoidectomy. As another example, the endoscopic device provides a greater field of view than conventional systems, which can increase safety.

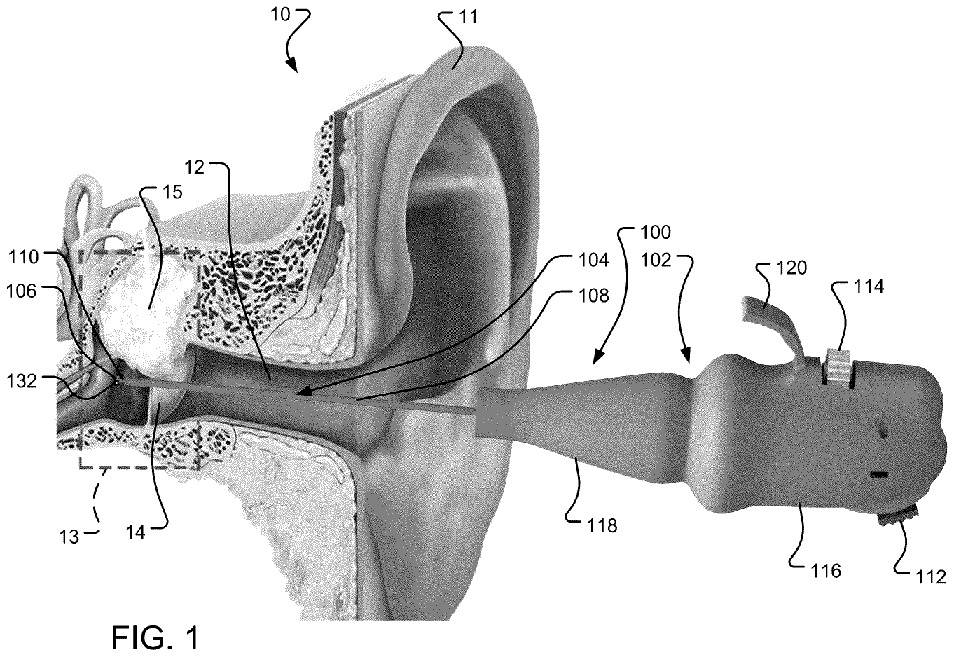

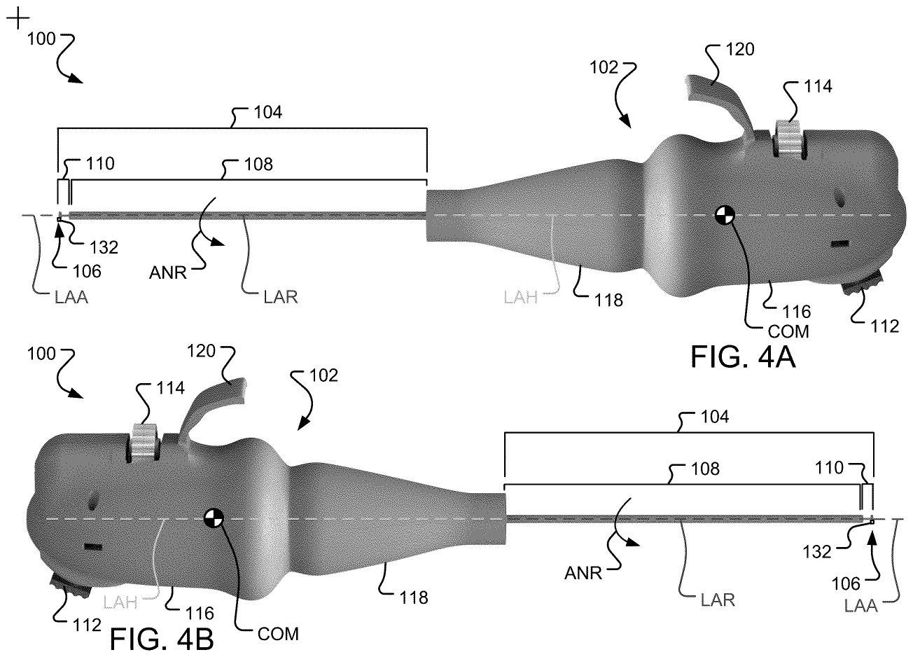

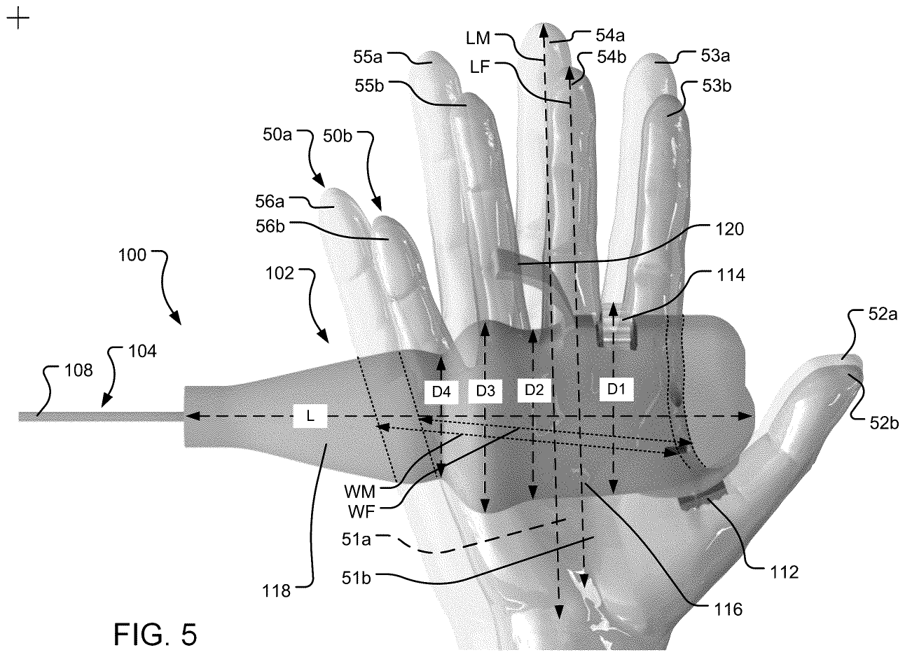

With reference to FIG. 1, an endoscopic device 100 is illustrated, according to one embodiment of the present disclosure. The endoscopic device 100 includes a handle 102 and a shaft 104 extending from the handle 102 to a distal end 106. An imaging sensor 132 is coupled to the shaft 104 at or near the distal end 106. It should be noted that the endoscopic device 100 is not necessarily illustrated to scale with respect to the ear 10 in FIG. 1. The shaft 104 is discussed in further detail, below, with respect to FIGS. 4A-4B, among others. The handle 102 is discussed in further detail, below, with respect to FIG. 5, among others.

The imaging sensor 132 is configured to provide viewing (e.g., imaging), such that the endoscopic device 100 can provide visualization within an ear 10 of a subject (e.g., person, patient). For example, the endoscopic device 100 can provide visualization of the middle ear 13 during an otolaryngology procedure. An operator (e.g., proceduralist, surgeon) can operate the endoscopic device 100.

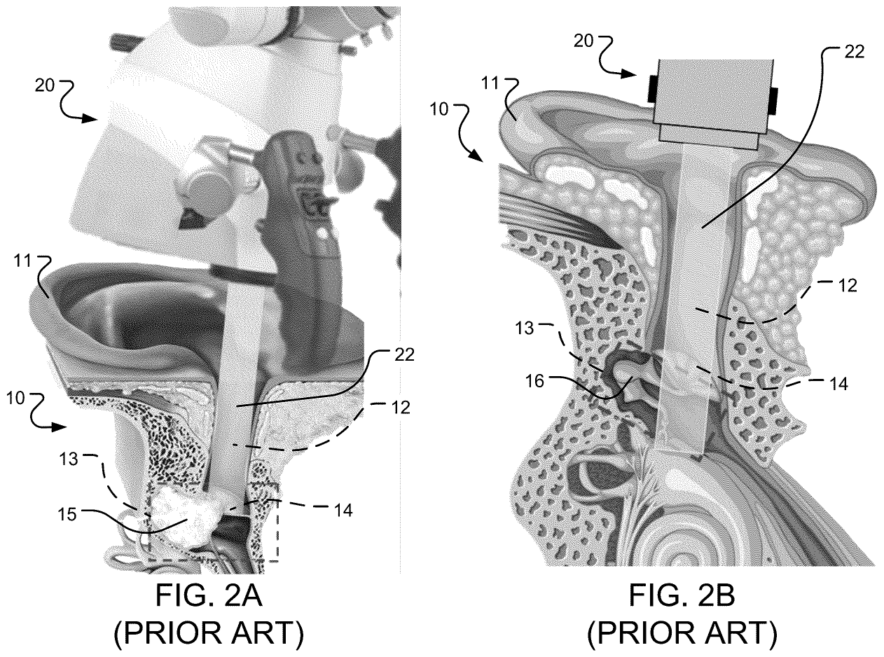

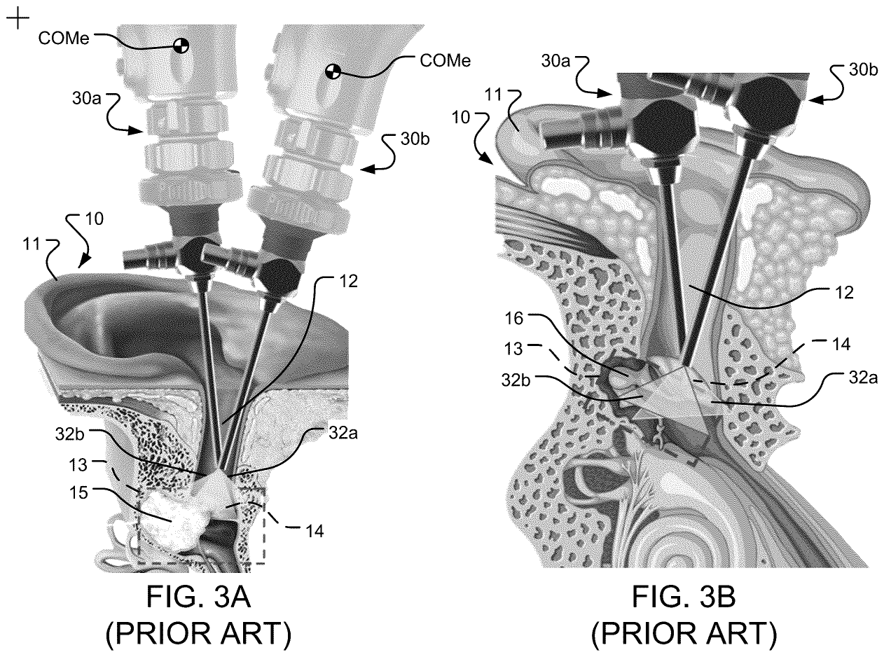

FIG. 1 shows an endoscopic device 100 being used to visualize an interior of an ear 10 of a patient. FIGS. 2A-2B depict a conventional floor-mounted microscope 30 being used to visualize an interior of an ear 10. FIGS. 3A-3B depict a conventional rigid endoscope 40 being used to visualize an interior of an ear 10. As depicted in FIGS. 1-3B, the ear 10 can include an auricle 11, an ear canal 12, and a middle ear 13.

Additionally, the ear 10 can include an eardrum 14 (also referred to as the tympanic membrane). In some instances, cholesteatoma 15 (as illustrated for example in FIG. 1, FIG. 2A, and FIG. 3A) can form within the middle ear 13. In particular, the cholesteatoma 15 can form in otomastoid cavities. In some examples, the cholesteatoma 15 attaches to the ossicles 16 (as illustrated for example in FIGS. 1, 2A, and 3A as can be understood from a comparison to FIGS. 2B and 3B), walls, and/or eardrum 18.

Turning to FIGS. 2A-2B, a conventional floor-mounted microscope 20 is illustrated in a perspective view and a more detailed perspective view, respectively. The floor-mounted microscope 20 provides a general field of view 22 (referred to as FOV), which is a single line-of-sight FOV. The floor-mounted microscope 20 can provide visualization of the middle ear 13 of the ear 10; however, the FOV 22 limits the visualization of the middle ear 13.

Turning to FIGS. 3A-3B, a conventional rigid endoscope 30 (e.g., rigid endoscope 30a, rigid endoscope 30b) is illustrated in a perspective view and a more detailed perspective view, respectively. The rigid endoscope 30 provides a general FOV 32 (e.g., FOV 32a, FOV 32b), which is generally between 70-degrees and 100-degrees. The rigid endoscope 30 can provide visualization of the middle ear 13 of the ear 10; however, the constraints of the ear canal 12 limits the visualization of the middle ear 13. That is, the range of motion of the rigid endoscope 30 (e.g., range of motion between a first position for a rigid endoscope 30a and a second position for a rigid endoscope 30b) is limited. As a result, the visualization of the middle ear 13 is closely related to the geometry of the ear canal 12.

In addition to having a limited FOV 32, the conventional rigid endoscope 30 includes a center of mass COMe (as illustrated in FIG. 3A). The center of mass COMe is above the backend camera of the rigid endoscope 30, which is generally above the portion of the rigid endoscope 30 that is gripped by the user (e.g., proceduralist, surgeon). In this manner, the rigid endoscope 30 can be cumbersome and difficult for the proceduralist to manipulate during a procedure (e.g., middle ear surgery).

Turning to FIGS. 4A-4B, the endoscopic device 100 is illustrated in a left-side view and a right-side view, respectively. The endoscopic device 100 includes a handle 102 and a shaft 104 extending from the handle 102 to a distal end 106 (also referred to as the distal tip). The shaft 104 includes a rigid portion 108 and an articulating portion 110. The rigid portion 108 extends distally from the handle 102 and the articulating portion 110 is extends distally from the rigid portion 108 towards the distal end 106 of the shaft 104. That is, the rigid portion 108 of the shaft 104 is between the handle 102 and the articulating portion 110 of the shaft 104.

In some instances, the shaft 104 (e.g., rigid portion 108) includes a lumen (not illustrated) extending therethrough. The lumen can receive surgical tools therein, such that the tools can be advanced through the lumen and extend towards a desired location (e.g., the middle ear 13 of the patient). Then, the tools can be used at the desired location.

Continuing with FIGS. 4A-4B, the rigid portion 108 defines a longitudinal axis LAR (also referred to as a first longitudinal axis). The longitudinal axis LAR of the rigid portion 108 is fixed with respect to the handle 102, such that movement (e.g., translation, rotation) of the handle 102 causes corresponding movement of the longitudinal axis LAR of the rigid portion 108. In some instances, the longitudinal axis LAR of the rigid portion 108 is parallel to a longitudinal axis LAH of the handle 102. In some instances, the longitudinal axis LAR of the rigid portion 108 is co-linear with the longitudinal axis LAH of the handle 102.

The rigid portion 108 is rigid, stiff, or otherwise unbending. In some instances, the rigid portion 108 is constructed of metal (e.g., surgical-grade stainless steel). Because the rigid portion 108 is rigid, the longitudinal axis LAR of the rigid portion 108 remains substantially straight or linear during use of the endoscopic device 100. Moreover, because the longitudinal axis LAR of the rigid portion 108 is fixed with respect to the handle 102, movement of the handle 102 causes corresponding movement of the rigid portion 108 of the shaft 104.

The rigid portion 108 can rotate about the longitudinal axis LAR. An angle of rotation ANR is defined about the longitudinal axis LAR. In some instances, the angle of rotation ANR is up to 360-degrees. That is, the angle of rotation ANR can be a full 360-degrees about the longitudinal axis LAR of the rigid portion 108. The rigid portion 108 can rotate clockwise (as illustrated in FIG. 4A) and/or counterclockwise (as illustrated on FIG. 4B). When the rigid portion 108 rotates about the longitudinal axis LAR, the articulating portion 110 (which is distal the rigid portion 108) correspondingly rotates about the longitudinal axis LAR.

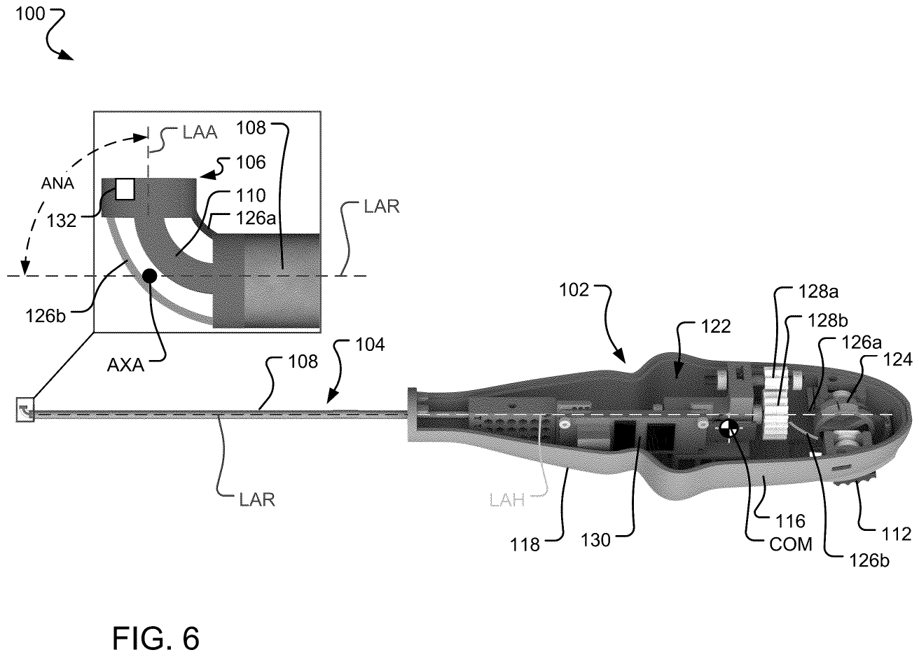

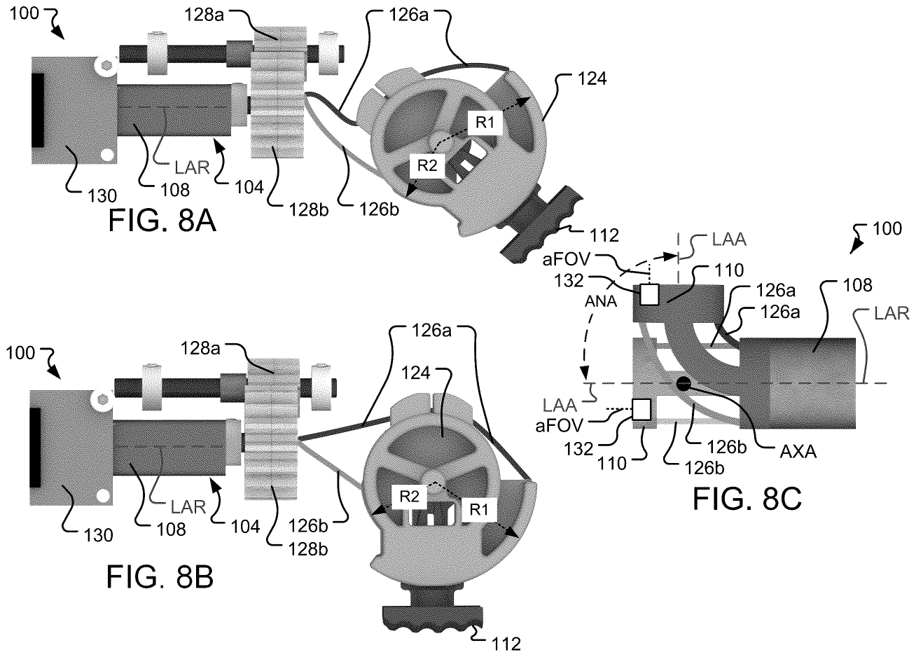

Continuing with FIGS. 4A-4B, the articulating portion 110 defines a longitudinal axis LAA (also referred to as a second longitudinal axis). In some instances, a living hinge is incorporated into the articulating portion 110. That is, the living hinge can articulate such that the articulating portion 110 is curved or otherwise nonlinear (as illustrated for example in FIG. 6 and FIG. 8C). Thus, the longitudinal axis LAA of the articulating portion 110 is defined by the distal end 106 of the articulating section 110 such that the longitudinal axis LAA is substantially straight or linear even though the articulating portion 110 is nonlinear. That is, the longitudinal axis LAA is normal to the distal end 106. In some instances, the articulating portion 110 is constructed of a polymer (e.g., surgical-grade silicone).

The articulating portion 110 can articulate with respect to the rigid portion 108.

For example, the articulating portion 110 can articulate about an axis of articulation AXA (which is illustrated for example in FIG. 6 and FIG. 8C). It should be noted that the axis of articulation AXA is illustrated as a “dot” because the axis of articulation extends into and out of the page in FIG. 6 and FIG. 8C. That is, the axis of articulation AXA is substantially perpendicular to both the longitudinal axis LAR of the rigid portion 108 and the longitudinal axis LAA of the articulating portion 110.

When the articulating portion 110 articulates, the articulating portion 110 transitions between an unarticulated position (as illustrated for example in FIG. 1, FIGS. 4A-4B, FIG. 7, and FIG. 8C (underlaid image)) and an articulated position (as illustrated for example in FIG. 6 and FIG. 8C (overlaid image)). An angle of articulation ANA is defined between the longitudinal axis LAR of the rigid portion 108 and the longitudinal axis LAA of the articulating portion 110. In the unarticulated position, the angle of articulation ANA can be approximately 0-degrees. In some instances, the longitudinal axis LAA of the articulating portion 110 is colinear to longitudinal axis LAR of the rigid portion 108.

In the articulated position, the angle of articulation ANA can be up to approximately 90-degrees. In certain instances, the angle of articulation can be up to approximately 80-degrees. In certain instances, the angle of articulation can be up to approximately 72-degrees. In certain instances, the angle of articulation can be up to approximately 70-degrees. In certain instances, the angle of articulation can be up to approximately 68-degrees. In some instances, in the articulated position, the longitudinal axis LAA of the articulating portion 110 is coplanar to longitudinal axis LAR of the rigid portion 108.

Continuing with FIGS. 4A-4B, the articulating portion 110 can rotate about the longitudinal axis LAR of the rigid portion 108. In some instances, the articulating portion 110 correspondingly rotates about the longitudinal axis LAR when the rigid portion 108 (which is proximal the articulating portion 110) rotates about the longitudinal axis LAR. That is, rotation of the rigid portion 108 about the longitudinal axis LAR causes rotation of the articulating portion 110 about the longitudinal axis LAR. In this manner, the articulating portion 110 can rotate to the angle of rotation ANR, which can be up to 360-degrees about the longitudinal axis LAR. When the longitudinal axis LAA of the articulating portion 110 is colinear to the longitudinal axis LAR of the rigid portion 108 (e.g., when the angle of articulation ANA is 0-degrees), the articulating portion 110 can rotate about the longitudinal axis LAA of the articulating portion 110.

The imaging sensor 132 is coupled to the articulating portion 110 of the shaft 104. Thus, when the articulating portion 110 articulates with respect to the rigid portion 108, the imaging sensor correspondingly articulates with respect to the rigid portion 108. Similarly, when the articulating portion 110 rotates about the longitudinal axis LAR of the rigid portion 108, the imaging sensor 132 correspondingly rotates about the longitudinal axis LAR. In some instances, the imaging sensor 132 is coupled at or near the distal end 106.

The articulating portion 110 can define a diameter. In some instances, the diameter is between approximately 3.5 mm and 4.3 mm. In some instances, the diameter is between approximately 3.6 mm and 4.2 mm. In some instances, the diameter is between approximately 3.7 mm and 4.1 mm. In some instances, the diameter is between approximately 3.8 mm and 4.0 mm. In some instances, the diameter is approximately 3.8 mm. In some instances, the diameter is approximately 3.9 mm. In some instances, the diameter is approximately 4.0 mm.

The articulating portion 110 can define a length. In some instances, the length is between approximately 6.8 mm and 7.6 mm. In some instances, the length is between approximately 6.9 mm and 7.5 mm. In some instances, the length is between approximately 7.0 mm and 7.4 mm. In some instances, the length is between approximately 7.1 mm and 7.3 mm. In some instances, the length is approximately 7.1 mm. In some instances, the length is approximately 7.2 mm. In some instances, the length is approximately 7.3 mm.

Turning to FIG. 5, the handle 102 (also referred to as an ergonomic handle) of the endoscopic device 100 is illustrated in a left-side view with a hand 50 (e.g., male hand 50a, female hand 50b) of a user. The handle 102 can include an articulation actuator 112 (also referred to as a first actuator) and a rotation actuator 114 (also referred to as a second actuator).

The articulation actuator 112 is configured to cause the articulating portion 110 (as illustrated in FIGS. 4A-4B) of the shaft 104 to articulate. That is, actuating the articulation actuator 112 causes the articulating portion 110 to articulate about the axis of articulation AXA (which is illustrated for example in FIG. 6 and FIG. 8C).

The rotation actuator 114 is configured to cause the articulating portion 110 (as illustrated in FIGS. 4A-4B) of the shaft 104 to rotate. That is, actuating the rotation actuator 114 causes the shaft 104 to rotate about longitudinal axis LAR of the rigid portion 108. In some instances, actuating the rotation actuator 114 causes the articulating portion 110 to rotate about longitudinal axis LAR of the rigid portion 108. In other instances, actuating the rotation actuator 114 causes the rigid portion 108 to rotate about longitudinal axis LAR, which correspondingly causes the articulating portion 110 to rotate about longitudinal axis LAR.

Continuing with FIG. 5, the handle 102 can be ergonomically configured such that it can be received and ergonomically gripped by a hand 50 (e.g., male hand 50a, female hand 50b) of a user (e.g., proceduralist, surgeon). For example, a male hand 50a in the 90th percentile of the population and a female hand 50b in the 90th percentile of the population are illustrated. The male hand 50a can define a width WM and a length LM. The width WM of the male hand 50a can be approximately 8.9 cm and the length LM of the male hand 50a can be approximately 19.3 cm. The female hand 50b can define a width WF and a length LF. The width WF of the female hand 50b can be approximately 7.9 cm and the length LF of the female hand 50b can be approximately 17.3 cm.

The hand 50 includes a palm 51 (e.g., male palm 51a, female palm 51b) and a thumb 52 (e.g., male thumb 52a, female thumb 52b) extending from the palm 51. Additionally, a forefinger 53 (e.g., male forefinger 53a, female forefinger 53b), a middle finger 54 (e.g., male middle finger 54a, female middle finger 54b), a ring finger 55 (e.g., male ring finger 55a, female ring finger 55b), and a little finger 56 (e.g., male little finger 56a, female little finger 56b) each extend from the palm 51.

Continuing with FIG. 5, the handle 102 can include a barrel portion 116 and a tapered portion 118 extending distally from the barrel portion 116. The barrel portion 116 can be configured to receive at least the thumb 52 and the forefinger 53 of the user when the user grips the handle 102. The center of mass COM of the handle 102 (as illustrated in FIGS. 4A-4B) can be in the barrel portion 116 of the handle 102. That is, the center of mass COM is within the portion gripped by the user (e.g., proceduralist, surgeon), which increases the users control of (or otherwise ability to manipulate) the endoscopic device 100. Additionally, both the articulation actuator 112 and the rotation actuator 114 can be positioned on the barrel portion 116 of the handle 102. In some instances, the articulation actuator 112 is located on the bottom side (also referred to as the first side) of the handle 102 and the rotation actuator 114 is located on the top side (also referred to as the second side) of the handle 102.

In some instances, the articulation actuator 112 is positioned on the handle 102 such that it can manipulated by the thumb 52 of the user. That is, the thumb 52 can actuate the articulation actuator 112 such that the articulating portion 110 articulates. In some instances, the articulation actuator 112 is a lever that is slidably actuated.

In some instances, the rotation actuator 114 is positioned on the handle 102 such that it can manipulated by the forefinger 53 of the user. That is, the forefinger 53 can actuate the rotation actuator 114 such that the articulating portion 110 rotates. In some instances, the rotation actuator 114 is a wheel that is rotatably actuated.

In some aspects, the user can operate the endoscopic device 100 with one hand 50. For example, the user can grip the handle 102 with one hand 50. Then, the user can move (e.g., translate, rotate) the handle 102 to cause corresponding movement of the rigid portion 108 of the shaft 104. The hand 50 of the user can actuate the articulation actuator 112 and/or rotation actuator 114 to cause the corresponding movement (e.g., articulation, rotation) of the articulating portion 110 of the shaft 104. In this manner, the opposite hand of the use is available to grip and manipulate (or otherwise use) medical tools, such as tools used during a middle ear procedure.

Continuing with FIG. 5, the barrel portion 116 can include a first portion defining a first diameter D1 (which can include the rotation actuator 114), a second portion defining a second diameter D2, a third portion defining a third diameter D3, and a fourth portion defining a fourth diameter D4. In some instances, the first portion, which is defined by diameter D1, is configured to receive the forefinger 53 of the user thereon. In some instances, the second portion, which is defined by diameter D2, is configured to receive middle finger 54 of the user thereon. In some instances, the outer surface of the barrel portion 116 is curved between each of the diameters (e.g., first diameter D1, second diameter D2, third diameter D3, fourth diameter D4).

Each diameter (e.g., first diameter D1, second diameter D2, third diameter D3, fourth diameter D4) can correspond to a cross-sectional area of the barrel portion 116. That is, the first diameter D1 can define a cross-sectional area that is larger than a cross-sectional area defined by the second diameter D2 and/or a cross-sectional area defined by the fourth diameter D4. Similarly, the third diameter D3 can define a cross-sectional area that is larger than a cross-sectional area defined by the second diameter D2 and/or a cross-sectional area defined by the fourth diameter D4. In some instances, first diameter D1 and third diameter D3 can be the same diameter.

In some instances, D1 is between approximately 5.6 cm and approximately 6.0 cm. For example, D1 can be approximately 5.8 cm. In some instances, D2 is between approximately 4.1 cm and approximately 4.5 cm. For example, D2 can be approximately 4.3 cm. In some instances, D3 is between approximately 5.6 cm and approximately 6.0 cm. For example, D3 can be approximately 5.8 cm. In some instances, D4 is between approximately 5.4 cm and approximately 5.8 cm. For example, D4 can be approximately 5.6 cm. In some cases, the maximum diameter of the barrel portion 116, which can be defined by the first diameter D1 and/or the third diameter D3, is between approximately 5.4 cm and approximately 6.2 cm.

The handle 102 defines a length L. In some instances, the length L is between approximately 15 cm and approximately 20 cm. In some instances, the length L is between approximately 16 cm and approximately 19 cm. In some instances, the length L is between approximately 17 cm and approximately 18 cm. In some instances, the length L is approximately 17.3 cm.

In some instances, a curvate member (also referred to as a finger guard) 120 extends from the barrel portion 116 of the handle 102. The curvate member 120 can extend over the second diameter D2, which is defined by the second portion that can receive the middle finger 54 of the user thereon. When a user grips the handle 102, the curvate member 120 can abut the hand 50 of the user (e.g., middle finger 54) such that the curvate member 120 stabilizes the handle 102 (and correspondingly the endoscopic device 100) with respect to the hand 50.

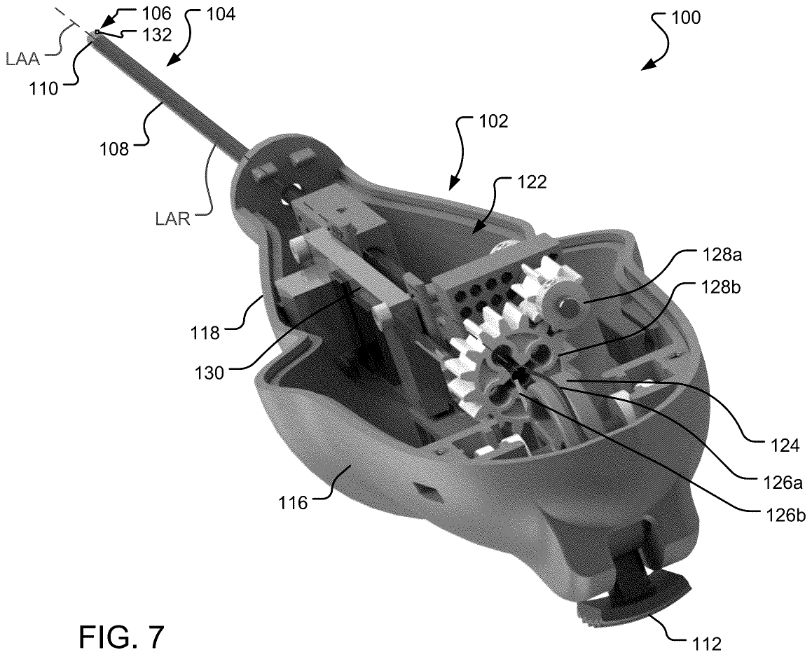

Turning to FIGS. 6-8C, various internal components of the endoscopic device 100 are illustrated. In FIGS. 6-7, an internal cavity 122 (and internal components contained therein) of the endoscopic device 100 are illustrated in a left-side, perspective view and a proximal-perspective view, respectively. For example, the internal cavity 122 can be defined by a shell of the handle 102. FIGS. 8A-8B illustrate the articulation actuator 112 in an unactuated position (FIG. 8A) and an actuated position (FIG. 8B) in left-side views. FIG. 8C illustrates the articulating portion 110 of the shaft 104 in an articulated position overlaying the unarticulated position.

As previously discussed, the articulation actuator 112 can be a lever. In some instances, the lever is coupled to a wheel 124 having one or more cables 126 (e.g., cable 126a, cable 126b) extending therefrom. In some instances, as illustrated in FIGS. 8A-8B, the wheel 124 defines a first radius R1 and a second radius R2 that is smaller than the first radius R1. For example, a first cable 126a can extend from a portion of the wheel 124 defined by the larger radius R1 and a second cable 126b can extend from a portion of the wheel 124 defined by the smaller radius R2. Each of the cables 126 extend to the articulating portion 110 of the shaft 104 (e.g., the distal end 106).

Actuation of the articulation actuator 112 (as illustrated in FIG. 8B with respect to the unactuated position in FIG. 8A), such as by sliding the lever with the thumb, causes the wheel 124 to rotate about an axis such that the first cable 126a (also referred to as the articulation cable) is tensioned. The tensioning of the first cable 126a causes the articulating portion 110 to transition to an articulated position. The larger radius R1 of the wheel 124 amplifies the angle of articulation ANA of the articulating section 110 relative to actuation (e.g., sliding) of the articulation actuator 112. That is, the larger radius R1 provides a mechanical advantage.

When the articulation actuator 112 (and corresponding wheel 124) is in an unactuated position (as illustrated in FIG. 8A), the articulating section 110 is correspondingly in an unarticulated position (as illustrated by the horizontal longitudinal axis LAA in FIG. 8C). When the articulation actuator 112 (and corresponding wheel 124) is in an actuated position (as illustrated in FIG. 8B), the articulating section 110 is correspondingly in an articulated position (as illustrated by the vertical longitudinal axis LAA in FIG. 8C). In some instances, the articulation actuator 112 is biased (e.g., spring loaded) to the unactuated position. That is, in the absence of a force (e.g., thumb force) applied to the articulation actuator 112, the articulating section 110 returns to (or otherwise remains in) an unarticulated position.

De-actuation of the articulation actuator 112 (as illustrated in FIG. 8A with respect to the actuated position in FIG. 8B), such as by sliding the lever with the thumb or by releasing the articulation actuator 112 (e.g., due to spring bias), causes the wheel 124 to rotate about an axis such that the second cable 126b (also referred to as the restoration cable) is tensioned. The tensioning of the second cable 126b causes the articulating portion 110 to transition to (e.g., be restored to) the unarticulated position.

The imaging sensor 132 has a field of view, which defines a field of view axis aFOV (as illustrated for example in FIG. 8C). Because the imaging sensor 132 is coupled to the articulating portion 110, the field of view axis aFOV of the imaging sensor 132 corresponds to (e.g., parallel to) the longitudinal axis LAA of the articulating section 110. Thus, movement (e.g., articulation, rotation) of the longitudinal axis LAA of the articulating section 110 causes corresponding movement of the field of view axis aFOV of the imaging sensor 132. In this manner, the field of view axis aFOV can be moved (e.g., articulated, rotated) with respect to the handle 102 of the endoscopic device 100.

The rotation actuator 114 can be wheel (e.g., gear), as illustrated for example in FIG. 1, FIGS. 4A-4B, and FIG. 5. The wheel of the rotation actuator 114 is in communication with the shaft 104. For example, the wheel can be in communication with one or more gears 128 (e.g., gear 128a, gear 128b) that are in communication with the shaft 104 (as illustrated for example in FIGS. 6-8B). For example, the wheel can be in communication with a first gear 128a (e.g., pinion gear) that is in communication with a second gear 128b. The rigid portion 108 of the shaft 104 is coupled to the second gear 128b such that rotation of the second gear 128b causes the rigid portion 108 to rotate about the longitudinal axis LAR. Rotation of the wheel causes the first gear 128a to rotate, which causes the second gear 128b to rotate. In this manner, when the rotation actuator 114 is rotated, the articulating portion 110 of the shaft 104 to rotates about the longitudinal axis LAR of the rigid section 108.

Continuing with FIGS. 6-8C, a printed circuit board 130 (referred to as a PCB) can be included in the internal cavity 122 of the endoscopic device 100. The PCB 130 is in communication with the imaging sensor 132. In this manner, the PCB 130 facilitates the capture of images such that the endoscopic device 100 can provide visualization. In some instances, the imaging sensor 132 is a camera (e.g., chip-on-tip camera). In some instances, the imaging sensor 132 has a light-emitting diode (LED) integrated therein.

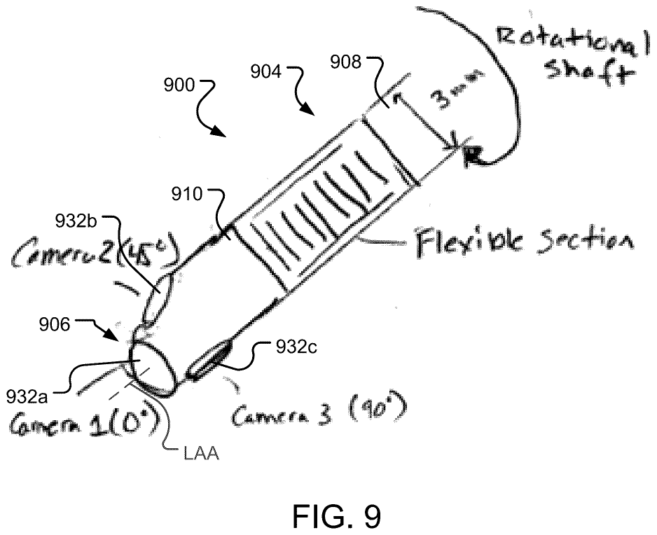

Turning to FIG. 9, the distal portion of a shaft 904 is illustrated in a perspective view, in accordance with embodiments of the disclosure. The shaft 904 can be included in an endoscopic device 900 that has one or more same or similar features as those described above with respect to the endoscopic device 100. Due to the same or similar features, the reference numbers and corresponding description provided above for various components, elements, portions, etc., included in the endoscopic device 100 (in FIG. 1 and FIGS. 4A-8C) may be generally applied to the same or similar components, elements, portions, etc., included in the endoscopic device 900 described in FIG. 9; however, the reference numbers in FIG. 9 are 900 series rather than 100 series. For example, the endoscopic device 900 illustrated in FIG. 9 can be used to provide visualization during a middle ear surgery in the same or similar manner as the endoscopic device 100.

The articulating portion 910 extends distally from the rigid portion 908 to the distal end 906. The articulating portion 910 can articulate with respect to the rigid portion 908 of the shaft 904. The articulating portion 910 includes two or more imaging sensors 932 (e.g., imaging sensor 932a, imaging sensor 932b, imaging sensor 932c), each of which are oriented at different angle with respect to the longitudinal axis LAA of the articulating portion 910. For example, a first imaging sensor 932a (first camera) can be oriented at approximately 0-degrees, a second imaging sensor 932b (second camera) can be oriented at approximately 45-degrees, and a third imaging sensor 932c (third camera) can be oriented at approximately 90-degrees. In some embodiments, the endoscopic device 900 can be used to access the middle ear and/or mastoid.

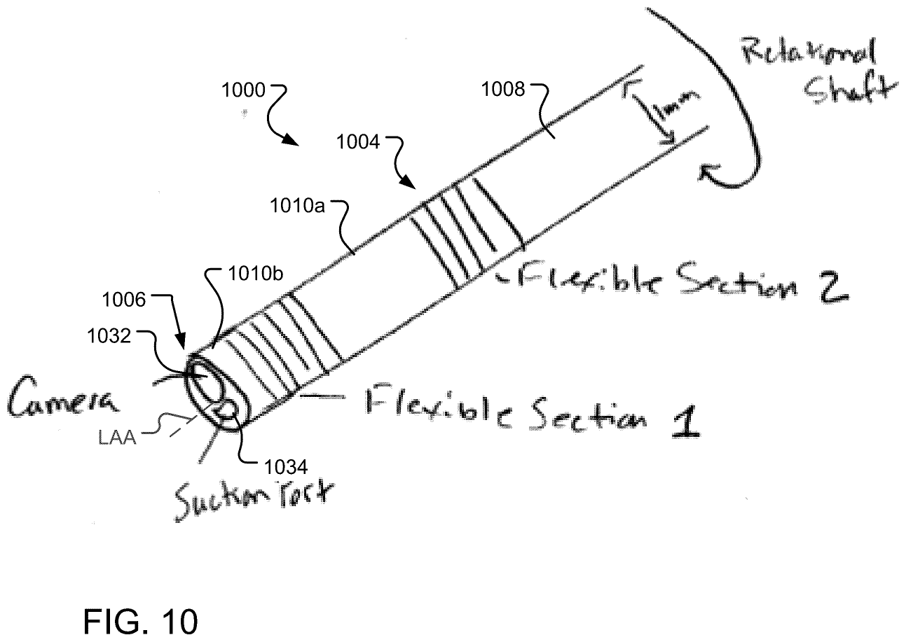

Turning to FIG. 10, the distal portion of a shaft 1004 is illustrated in a perspective view, in accordance with embodiments of the disclosure. The shaft 1004 can be included in an endoscopic device 1000 that has one or more same or similar features as those described above with respect to the endoscopic device 100. Due to the same or similar features, the reference numbers and corresponding description provided above for various components, elements, portions, etc., included in the endoscopic device 100 (in FIG. 1 and FIGS. 4A-8C) may be generally applied to the same or similar components, elements, portions, etc., included in the endoscopic device 1000 described in FIG. 10; however, the reference numbers in FIG. 10 are 1000 series rather than 100 series. For example, the endoscopic device 1000 illustrated in FIG. 10 can be used to provide visualization during a middle ear surgery in the same or similar manner as the endoscopic device 100.

A first articulating portion 1010a extends distally from the rigid portion 1008. A second articulating portion 1010b extends distally from the first articulating portion 1010a to the distal end 1006. The first articulating portion 1010a can articulate with respect to the rigid portion 1008 of the shaft 904. The second articulating portion 1010b can articulate with respect to the first articulating portion 1010a. The second articulating portion 1010a includes an imaging sensor 1032 (camera) coupled to the distal end 1006. The shaft 1004 includes a lumen 1034 extending therethrough (e.g., through the distal end 1006). The lumen 1034 can define a suction port that can be used for suction. In some embodiments, the endoscopic device 1000 can be used to access the inner ear.

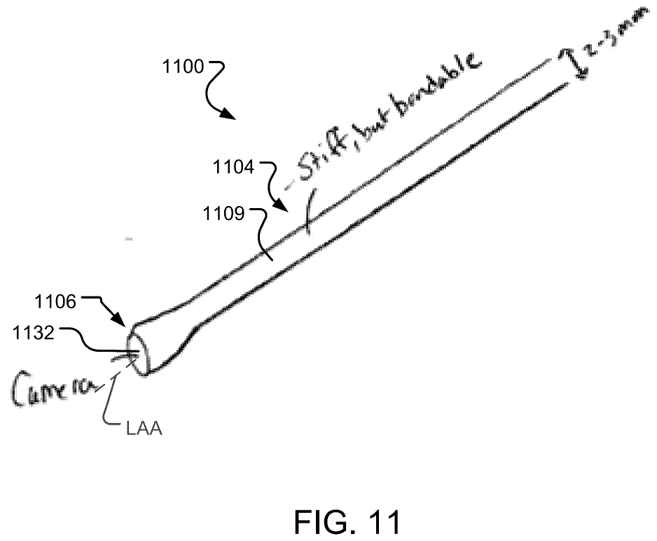

Turning to FIG. 11, the distal portion of a shaft 1104 is illustrated in a perspective view, in accordance with embodiments of the disclosure. The shaft 1104 can be included in an endoscopic device 1000 that has one or more same or similar features as those described above with respect to the endoscopic device 100. Due to the same or similar features, the reference numbers and corresponding description provided above for various components, elements, portions, etc., included in the endoscopic device 100 (in FIG. 1 and FIGS. 4A-8C) may be generally applied to the same or similar components, elements, portions, etc., included in the endoscopic device 1100 described in FIG. 11; however, the reference numbers in FIG. 11 are 1100 series rather than 100 series. For example, the endoscopic device 1100 illustrated in FIG. 11 can be used to provide visualization during a middle ear surgery in the same or similar manner as the endoscopic device 100.

A semi-rigid portion 1109 extends to the distal end 1106 of the shaft 1104. The semi-rigid portion 1109 is relatively stiff, but also bendable. That is, the longitudinal axis LAA of the semi-rigid portion can be bent, curved, or otherwise manipulated. The semi-rigid portion 1109 includes an imaging sensor 1132 (camera) coupled to the distal end 1106. In some embodiments, the endoscopic device 1100 can be used to as an intubation stylet.

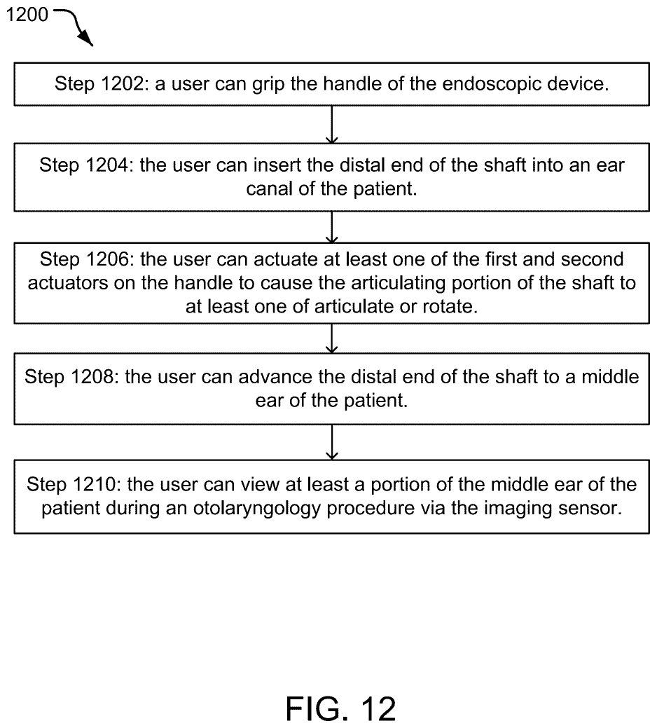

Turning to FIG. 12, an example of a method 1200 of using the endoscopic device 100 is provided in a flowchart. The endoscopic device 100 can provide visualization during a middle ear surgery (e.g., an otolaryngology procedure) of a patient. At step 1202, a user (e.g., proceduralist, surgeon) can grip the handle 102 of the endoscopic device 100 with a hand 50 (see e.g., FIG. 5). At step 1204, the user can insert the distal end 106 of the shaft 104 into an ear canal 12 of the patient (see e.g., FIG. 1). At step 1206, the user can actuate at least one of the actuators (e.g., articulation actuator 112, rotation actuator 114) on the handle 102 to cause the articulating portion 110 of the shaft 104 to at least one of articulate or rotate. At step 1208, the user can advance the distal end 106 of the shaft 104 to a middle ear 13 of the patient (see e.g., FIG. 1). At step 1210, the user can view at least a portion of the middle ear 13 of the patient during an otolaryngology procedure via the imaging sensor 132.

### EXAMPLES

Various tests were conducted to evaluate the performance of the endoscopic device 100.

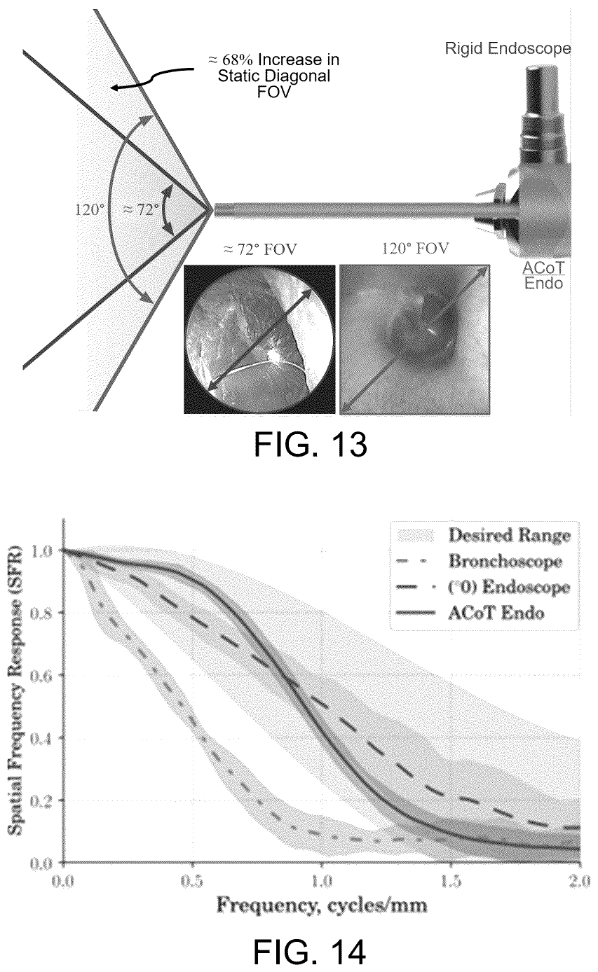

FIG. 13 illustrates the endoscopic device 100 (the ACoT Endo) diagonal field of view compared to a standard rigid endoscope. The ACoT Endo's diagonal field of view increased by appropriately 69% compared to the 0° Karl Storz endoscope, and it changed the typical shape of the view from a circle to a square. This change in shape allows the user to see more of the surrounding tissue which can increase safety and efficiency during procedures.

FIG. 14 illustrates the Spatial Frequency Response (SFR) comparison of 20 averaged samples from the 0° Karl Storz endoscope, Ambu bronchoscope, and the endoscopic device 100 (the ACoT Endo). The shaded region for each endoscope is ±2 σ from the average SFR plotted and encompasses 95% of the data. The desired region for the ACoT Endo to fall in between is ±4 σ from the 0° Storz endoscope. This comparison shows that the camera system on the ACoT Endo is within the desired range for capturing high and low details.

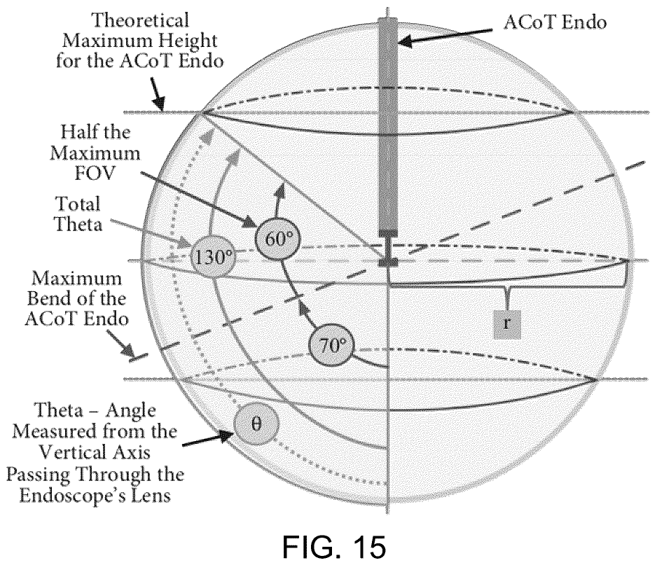

FIG. 15 illustrates a visual representation of variables used in the equation A=2πr2(1−cos θ). To quantify the advantage of the controllable degrees of freedom (DOFs), tests were conducted to compare the view space of the endoscopic device (the ACoT Endo) from a fixed point in a cavity. A uniform sphere was used, which allowed the equation (above) to be used to calculate the surface area (SA) that can be seen by an endoscope. The variables for this equation are illustrated in FIG. 15 where theta is the angle measured from the vertical axis passing through the endoscope's lens and r is the radius of the sphere. Theta for the ACoT Endo comprises half the maximum FOV and the maximum angle it could bend. The standard rigid endoscopes cannot bend, so their theta will be a product of their FOV only. The 0° and 30° Storz endoscopes have a FOV of 71° while the 45° Storz endoscope has a maximum FOV of 100°. The ACoT Endo is able to visualize approximately 775%, 116%, and 48% more surface area compared to the standard 0°, 30°, and 45° Storz rigid endoscopes, respectively.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements and methods which, although not explicitly shown or described herein, embody the principles of the invention and are thus within the spirit and scope of the present invention. From the above description and drawings, it will be understood by those of ordinary skill in the art that the particular embodiments shown and described are for purposes of illustrations only and are not intended to limit the scope of the present invention. References to details of particular embodiments are not intended to limit the scope of the invention.

What is claimed is:

1. An endoscopic device comprising:
a handle;
a shaft extending from the handle to a distal end, the shaft including:
a rigid portion distal the handle and defining a first longitudinal axis;
an articulating portion distal the rigid portion and extending distally towards the distal end, the articulating portion defining a second longitudinal axis, the articulating portion configured to articulate about an axis of articulation, wherein the axis of articulation is substantially perpendicular to each of the first longitudinal axis and the second longitudinal axis; and
an imaging sensor coupled to the shaft at least near, if not on, the distal end.

2. The endoscopic device of claim 1, wherein the second longitudinal axis is co-linear with the first longitudinal axis when the articulating portion is not articulated, wherein the second longitudinal axis is coplanar with the first longitudinal axis when the articulating portion is articulated.

3. The endoscopic device of claim 1, wherein the second longitudinal axis defines an angle of articulation with respect to the first longitudinal axis, wherein the angle of articulation is up to approximately 90-degrees.

4. The endoscopic device of claim 1, wherein the shaft includes a living hinge between the rigid portion and the articulating portion, the living hinge defining the axis of articulation.

5. The endoscopic device of claim 1, wherein the articulating portion is configured to rotate about the first longitudinal axis.

6. The endoscopic device of claim 5, wherein rotation of the articulating portion about the first longitudinal axis defines an angle of rotation, wherein the angle of rotation is up to 360-degrees.

7. The endoscopic device of claim 1, wherein the handle includes a first actuator and a second actuator, wherein actuating the first actuator causes the articulating portion to articulate about the axis of articulation, wherein actuating the second actuator causes the shaft to rotate about the first longitudinal axis.

8. The endoscopic device of claim 1, wherein the handle is ergonomically configured such that when the handle is gripped, the shaft extends downward, a first actuator is positioned for thumb-manipulation and a second actuator is positioned for forefinger-manipulation.

9. An endoscopic device adapted to be ergonomically gripped by a hand of a user, the hand including a palm, a thumb, a forefinger, a middle finger, a ring finger and a little finger, these four fingers and the thumb extending from the palm, the endoscopic device comprising:
an ergonomic handle configured to be received by the hand of the user, the ergonomic handle including first and second actuators;
a shaft extending distally from the ergonomic handle, the shaft including a rigid portion and an articulating portion; and
an imaging sensor coupled to the articulating portion of the shaft,
wherein the first actuator is configured to cause the articulating portion to articulate with respect to the rigid portion, and the second actuator is configured to cause the articulating portion to rotate about a longitudinal axis of the rigid portion,
wherein, when the ergonomic handle is gripped by the hand of the user, the palm and at least two of the four fingers wrap around the ergonomic handle, the thumb being positioned to manipulate the first actuator, the forefinger positioned to manipulate the second actuator, and the shaft extending downward away from the ergonomic handle and the first actuator.

10. The endoscopic device of claim 9, wherein the ergonomic handle includes a barrel portion and a tapered portion extending distally from the barrel portion, the barrel portion configured to receive at least the forefinger, middle finger and thumb of the user thereon.

11. The endoscopic device of claim 10, wherein the barrel portion includes a first portion defining a first cross-sectional area and a second portion defining a second cross-sectional area that is smaller than the first cross-sectional area, wherein the first portion is configured to receive the forefinger of the user thereon and the second portion is configured to receive the middle finger of the user thereon.

12. The endoscopic device of claim 9, wherein the articulating portion is configured to articulate about an axis of articulation that is substantially perpendicular to a longitudinal axis of the rigid portion.

13. The endoscopic device of claim 9, wherein the first actuator is on a first side of the ergonomic handle and the second actuator is on a second side of the ergonomic handle opposite the first side.

14. The endoscopic device of claim 9, wherein the first actuator is positioned for thumb-manipulation and the second actuator is positioned for forefinger-manipulation.

15. The endoscopic device of claim 9, wherein the first actuator includes a lever configured to receive the thumb of the user thereon such that the lever can be slidably actuated, wherein the second actuator includes a wheel configured to receive the forefinger of the user thereon such that the wheel can be rotatably actuated.

16. The endoscopic device of claim 9, wherein the ergonomic handle defines a length and a barrel portion of the ergonomic handle defines a maximum diameter, wherein the length is between approximately 15 cm and approximately 20 cm, wherein the maximum diameter is between approximately 5.4 cm and approximately 6.2 cm.

17. A method of using an endoscopic device to provide visualization during a middle ear surgery of patient, the method comprising:
gripping a handle of the endoscopic device with a hand, the endoscopic device including the handle, a shaft distally extending from the handle to a distal end of the shaft and an imaging sensor at least near, if not on, the distal end, the shaft including a rigid portion and an articulating portion distal the rigid portion, the handle including first and second actuators, the first actuator configured to cause the articulating portion to articulate with respect to the rigid portion and the second actuator configured to cause the articulating portion to rotate with respect to a longitudinal axis of the rigid portion;
inserting the distal end of the shaft into an ear canal of an ear of the patient; and
actuating at least one of the first and second actuators on the handle to cause the articulating portion of the shaft to at least one of articulate or rotate.

18. The method of claim 17, wherein gripping the handle of the endoscopic device with the hand results in a palm, a forefinger and a middle finger, all three of which are associated with the hand, wrapping around the handle, a thumb associated with the hand positioned to manipulate the first actuator, the forefinger positioned to manipulate the second actuator, and the shaft extending downward away from the handle and the first actuator.

19. The method of claim 17, further comprising advancing the distal end to a middle ear of the patient.

20. The method of claim 17, further comprising viewing at least a portion of a middle ear of the patient during otolaryngology procedure, wherein viewing is provided via the imaging sensor.
